(19) United States
(12) Patent Application Publication
(10) Pub. No.: US 2026/0221220 A1
(43) Pub. Date: Jul. 30, 2026

(54) GENERATION OF BIOSYNTHETIC ASSEMBLY LINES WITH GENOMIC A.I.

(71) Applicant: Tatta Bio, Tiburon, CA (US)

(72) Inventors: Yunha Hwang, Brooklyn, NY (US); Andre Liang Comman, Brooklyn, NY (US)

(21) Appl. No.: 19/460,583

(22) Filed: Jan. 27, 2026

Related U.S. Application Data
(60) Provisional application No. 63/750,100, filed on Jan. 27, 2025.

(51) Int. Cl. G16B 15/30; G16B 5/00; G16B 15/20; G16B 40/20

(52) U.S. Cl. CPC G16B 15/30; G16B 5/00; G16B 15/20; G16B 40/20

(57) ABSTRACT

The present disclosure relates to a method for designing biosynthetic assembly lines using a chemistry-aware biosynthetic assembly line (CABAL) decoder. The method involves providing a CABAL decoder that includes a chemical-to-protein model designed to create a biosynthetic assembly line based on a specified target desired chemistry. Upon receiving a target desired chemistry as input, the CABAL decoder generates a biosynthetic assembly line design. The generated biosynthetic assembly line design is then outputted, facilitating the creation of tailored biosynthetic pathways for specific chemical transformations.

### CROSS-REFERENCE TO RELATED APPLICATION(S

This application claims priority to, and the benefit of, co-pending United States Provisional Application 63/750,100, filed January 27, 2025, for all subject matter common to both applications. The disclosure of said provisional application is hereby incorporated by reference in its entirety.

### Statement Regarding Federally Sponsored Research or Development

This invention was made with U.S. Government support under Agreement No. HR00112530038 awarded by Defense Advanced Research Projects Agency. The U.S. Government has certain rights in the invention.

### FIELD OF THE INVENTION

The present invention relates to designing biosynthetic assembly lines for producing chemical compounds. In particular, the present invention relates to using genomic artificial intelligence (A.I.) for the generation of biosynthetic assembly lines.

### BACKGROUND

Conventional (systems, devices, methods) have or implement processes of designing biosynthetic assembly lines by 1) identifying template loci for engineering 2) identifying modules and fusion sites 3) combinatorially adding/rearranging/deleting modules to yield novel chimeric biosynthetic assembly lines.

However, this (technology, device, system, methodology, etc.) experiences some shortcomings. 1) it is very slow and manual, 2) often unsuccessful due to incompatibility between biosynthesis domains and reactions (gate-keeping and module skipping effects). 3) low product yield due to incompatibility with the expression host system.

Chemical synthesis of novel compounds is time-consuming and often unsuccessful. For example, it took nine years to artificially synthesize Erythromycin. While generative AI in molecular structures have made great progress in the past few years, many of these in silico molecules cannot be synthesized resulting in slow iteration and validation, and overall reduced utility of these methods.

With advances in bioprospecting and metagenomics, our ability to discover the assembly machinery for synthesizing novel classes of bioactive compounds have increased considerably. However, meaningfully modifying these novel classes of compounds requires engineering the genes involved in the step-by-step chemical transformation. Due to the complex nature of these multi-domain, multi-protein loci, previous efforts for engineering biosynthetic assembly lines have resulted in failures or poor product yields. While recent advances in evolution-guided assembly line rational engineering have shown promise, the full extent of biophysical and functional constraints in assembly line design evades human characterization. This results in labor-intensive iteration cycles with low success rates.

FIG. 1 depicts a high-level diagram 100 of the steps involved in the current process for synthesizing a desired compound 102 by engineering a biosynthetic assembly line 104. This involves identification of a template assembly line (step 106). Here, one must first identify an engineerable and well-characterized loci with a known product and catalytic modules. Then Retrosynthesis-based identification of reaction steps to modify is performed (step 108). Retrosynthesis software can be used to predict the modifications in the reaction steps. Identification of fusion sites in template loci (step 110) and identification of candidate modules from the database (step 112) is then performed for in silico design of fusion loci. This results in the expression of the designed loci and validation of yielded product (step 114). Due to poor database curation, limitations in retrosynthesis software, and incomplete

understanding of the assembly line logic and biophysical constraints, each of the above steps require expert scrutiny and trial-and-errors.

### SUMMARY

There is a need for rapidly and computationally designing genomic regions encoding biosynthetic assembly lines given a desired set of reaction modules. The present invention is directed toward further solutions to address this need, in addition to having other desirable characteristics. Specifically, the application of genomic language modeling and generative AI to design biosynthetic pathways to result in novel biochemistry.

In accordance with embodiments of the present invention, a method of designing biosynthetic assembly lines is provided. The method involves providing a chemistry-aware biosynthetic assembly line (CABAL) decoder comprising a chemical-to-protein model configured to design a biosynthetic assembly line conditioned upon a target desired chemistry; receiving a target desired chemistry as input; generating, using the CABAL decoder, a biosynthetic assembly line ; and outputting the generated biosynthetic assembly line.

In accordance with aspects of the present invention, the chemistry-aware biosynthetic assembly line (CABAL) decoder is configured for multi-domain and multi-protein design.

In accordance with aspects of the present invention, the CABAL decoder is trained to comprise a latent understanding of biochemistry by learning a relationship between chemical products and catalytic modules.

In accordance with aspects of the present invention, the provided chemistry-aware biosynthetic assembly line (CABAL) decoder is created by: training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and

training the BAL-decoder on a curated dataset of chemical structure-biosynthetic assembly line pairs allowing for conditioning of sequence generation with target desired chemistry to create a chemistry-aware biosynthetic assembly line (CABAL) decoder. In some such aspects, the genomic language model (gLM) decoder is trained on a multi-modal Open MetaGenomic (OMG) corpus. In other such aspects, performance of gLM-decoder is validated using in silico protein design validation methods, by structure-based quantification of stability and foldability of protein-protein complexes and domain-domain interactions. In still other such aspects, the BAL-decoder is configured to identify fusion sites, linker sequences, and catalytic modules and their substrate and stereo-specificities. In further such aspects, the BAL-decoder is validated using predicted structural plausibility and detection of catalytic domains and their substrate and stereo-specificities. In still further such aspects, the chemistry-aware biosynthetic assembly line (CABAL) decoder is configured to learn relationships between chemical structures and biosynthetic module arrangements. In other such aspects, the chemistry-aware biosynthetic assembly line (CABAL) decoder is validated using a curated held-out validation set.

In accordance with aspects of the present invention, the target desired chemistry involves a desired chemical compound and the generated biosynthetic assembly line design is capable of catalyzing the desired chemical compound.

In accordance with aspects of the present invention, the target desired chemistry involves desired enzymatic domains with their substrate and stereo-specificities and the generated biosynthetic assembly line design comprises the enzymatic domains with their substrate and stereo-specificities.

In accordance with aspects of the present invention, the desired target chemistry involves a template biosynthetic assembly line that needs to be optimized or modified and the generated biosynthetic assembly line design comprises an optimized or modified biosynthetic assembly line.

In accordance with aspects of the present invention, the method further involves training the chemical-to-protein model using one or more of: a retrieval augmented biosynthetic assembly line dataset and a train-test split paired-dataset of biosynthetic assembly lines and chemical products.

In accordance with embodiments of the present invention, a system for designing biosynthetic assembly lines is provided, the system involves a chemistry-aware biosynthetic assembly line (CABAL) decoder comprising a chemical-to-protein model configured to design a biosynthetic assembly line conditioned upon a target desired chemistry and a processor. The processor is configured to: receive a target desired chemistry as input; generate, using the CABAL decoder, a biosynthetic assembly line; and output the generated biosynthetic assembly line design.

In accordance with aspects of the present invention, the chemistry-aware biosynthetic assembly line (CABAL) decoder is configured for multi-domain and multi-protein design.

In accordance with aspects of the present invention, the CABAL decoder is trained to comprise a latent understanding of biochemistry by learning a relationship between chemical products and catalytic modules.

In accordance with aspects of the present invention, the chemistry-aware biosynthetic assembly line (CABAL) decoder is created by: training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder. In some such aspects, the genomic language model (gLM) decoder is trained on a multi-modal Open MetaGenomic (OMG) corpus. In other such aspects, performance of gLM-decoder is validated using in silico protein design validation methods, by structure-based quantification of stability and foldability of protein-protein complexes and domain-domain interactions. In still other such aspects, the BAL-decoder is configured to identify fusion sites, linker sequences, and catalytic modules and their substrate and stereo-specificities. In further such aspects, the BAL-decoder is validated using predicted structural plausibility and detection of catalytic domains and their substrate and stereo-specificities. In still further such aspects, the chemistry-aware biosynthetic assembly line (CABAL) decoder is configured to learn

relationships between chemical structures and biosynthetic module arrangements. In other such aspects, the chemistry-aware biosynthetic assembly line (CABAL) decoder is validated using a curated held-out validation set.

In accordance with aspects of the present invention, the target desired chemistry involves a desired chemical compound and the generated biosynthetic assembly line design is capable of catalyzing the desired chemical compound.

In accordance with aspects of the present invention, the target desired chemistry involves desired enzymatic domains with their substrate and stereo-specificities and the generated biosynthetic assembly line design is capable of catalyzing the enzymatic domains with their substrate and stereo-specificities.

In accordance with aspects of the present invention, the desired target chemistry involves a template biosynthetic assembly line that needs to be optimized or modified and the generated biosynthetic assembly line design comprises an optimized or modified biosynthetic assembly line.

In accordance with aspects of the present invention, the processor is further configured to train the chemical-to-protein model using one or more of: a retrieval augmented biosynthetic assembly line dataset and a train-test split paired-dataset of biosynthetic assembly lines and chemical products.

In accordance with embodiments of the present invention, a method of generating a chemistry-aware biosynthetic assembly line (CABAL) decoder is provided. The method involves training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder.

In accordance with aspects of the present invention, the genomic language model (gLM) decoder is trained on a multi-modal Open MetaGenomic (OMG) corpus.

In accordance with aspects of the present invention, performance of gLM-decoder is validated using in silico protein design validation methods, by structure-based quantification of stability and foldability of protein-protein complexes and domain-domain interactions.

In accordance with aspects of the present invention, the BAL-decoder is configured to identify fusion sites, linker sequences, and catalytic modules.

In accordance with aspects of the present invention, the BAL-decoder is validated using predicted structural plausibility and detection of catalytic domains and their substrate and stereo-specificities.

In accordance with aspects of the present invention, the chemistry-aware biosynthetic assembly line (CABAL) decoder is configured to learn relationships between chemical structures and biosynthetic module arrangements.

In accordance with aspects of the present invention, the chemistry-aware biosynthetic assembly line (CABAL) decoder is validated using a curated held-out validation set.

In accordance with aspects of the present invention, the target desired chemistry comprises one or more of a desired chemical compound, desired enzymatic domains with their substrate and stereo-specificities, and a template biosynthetic assembly line that needs to be optimized or modified.

In accordance with embodiments of the present invention, a chemical-to-protein model configured to design a biosynthetic assembly line conditioned upon a target desired chemistry is provided. The chemical-to-protein model is created by a process involving training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a

BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder.

In accordance with aspects of the present invention, the genomic language model (gLM) decoder is trained on a multi-modal Open MetaGenomic (OMG) corpus.

In accordance with aspects of the present invention, performance of gLM-decoder is validated using in silico protein design validation methods, by structure-based quantification of stability and foldability of protein-protein complexes and domain-domain interactions.

In accordance with aspects of the present invention, the BAL-decoder is configured to identify fusion sites, linker sequences, and catalytic modules and their substrate and stereo-specificities.

In accordance with aspects of the present invention, the BAL-decoder is validated using predicted structural plausibility and detection of catalytic domains and their substrate and stereo-specificities.

In accordance with aspects of the present invention, the chemistry-aware biosynthetic assembly line (CABAL) decoder is configured to learn relationships between chemical structures and biosynthetic module arrangements.

In accordance with aspects of the present invention, the chemistry-aware biosynthetic assembly line (CABAL) decoder is validated using a curated held-out validation set.

In accordance with aspects of the present invention, the target desired chemistry comprises one or more of a desired chemical compound, desired enzymatic domains with their substrate and stereo-specificities, and a template biosynthetic assembly line that needs to be optimized or modified.

The present invention makes use of a chemical to protein model to replace step 106 through step 114 of the conventional process 100 shown in FIG. 1. This approach is a fundamental advance from existing conventional protein sequence design models (e.g., ESM3, ProGen2) for the following reasons:

The present invention is capable of multi-domain, multi-protein design. Existing protein design tools are not capable of learning domain-domain, protein-protein interactions (PPIs), because they are trained on single proteins with limited context length. To date, there is no protein sequence model that is capable of generating multi-domain, multi-protein sequences at once. It was previously shown that multi-protein information can be learned and leveraged for decoding using genomic language models (gLMs).

The present invention is designed to be chemistry-aware. The disclosed model has a latent understanding of biochemistry by learning the relationship between chemical products and the substrate and stereo-specificities of the catalytic modules encoded in their corresponding biological sequences. In doing so, desired chemistry can serve as the conditioning signal for biosynthetic assembly line generation.

The present invention provides a technical leap for the field of generative AI for chemistry, by providing an avenue to synthesize diverse sets of previously unsynthesizable molecules.

The present invention enables a user to specify desired substrate and/or stereo-specificities of catalytic modules, which allows controllable sequence design that cannot be achieved with sequence mining.

The present invention further enables biosynthetic assembly line refinement using the chemistry-aware nature of the model. This enables the model to design assembly line sequences that can carry out only chemically possible and compatible reactions given the substrates and desired product.

The present invention increases the success rate of molecular synthesis with de novo sequence designs and therefore reduces the experimental labor and DNA synthesis cost needed for commercialization-ready sequence designs.

### BRIEF DESCRIPTION OF THE FIGURES

These and other characteristics of the present invention will be more fully understood by reference to the following detailed description in conjunction with the attached drawings, in which:

FIG. 1 is a high-level diagram of the current process for synthesizing a desired compound by engineering a biosynthetic assembly line;

FIG. 2 is a high-level diagram of the process for synthesizing a desired compound by engineering a biosynthetic assembly line in accordance with embodiments of the present invention;

FIG. 3 is a flow diagram 300 for a method of designing biosynthetic assembly lines in accordance with embodiments of the present invention;

FIG. 4 is a flow diagram for a method of generating a chemistry-aware biosynthetic assembly line (CABAL) decoder in accordance with embodiments of the present invention;

FIG. 5 is a diagram 500 depicting the hierarchical training of the generative artificial intelligence (A.I.) models with an increasing degree of specialization in accordance with embodiments of the present invention; and

FIG. 6 is a diagrammatic illustration of a high-level architecture for implementing the invention in accordance with embodiments of the present invention.

### DETAILED DESCRIPTION

An illustrative embodiment of the present invention relates to designing biosynthetic assembly lines for producing chemical compounds using genomic artificial intelligence.

As used herein, a "genomic language models " or "gLM" refers to a type of artificial intelligence model that adapts large language models (LLMs) to "read" and understand DNA sequences like text, treating them as a language to predict functions, identify variants, and even design new sequences, bridging gaps between sequence and organismal function, with applications from tracking viral evolution (like SARS-CoV-2) to accelerating drug discovery, though challenges remain in explaining complex individual variations. Large Language models (LLMs) are part of the class of computational models known as foundation models. An LLM is a neural network-based system pre-trained on extensive datasets of textual materials, including books, articles, websites, and other written content. The training enables the LLM to process, understand, and generate human language, including grammar, syntax, context, and semantic relationships.

In certain embodiments, an LLM utilizes a transformer architecture comprising one or more neural network layers that employ self-attention mechanisms to process natural language inputs. The transformer architecture may include encoders, decoders, or encoder-decoder configurations that perform operations on input data to generate corresponding output data. The LLM processes natural language by transforming text into token embeddings (numerical representations of linguistic units such as words, subwords, or characters), estimating positional relationships between tokens, and determining contextual relationships between tokens using self-attention mechanisms.

An LLM is characterized by its scale, typically containing a large number of trainable parameters, often ranging from millions to billions of parameters. The parameter count enables the LLM to capture complex language patterns and perform a wide range of natural language processing tasks. Examples of LLMs include, but are not limited to, Generative Pre-trained Transformer models (GPT), Bidirectional Encoder Representations from Transformers (BERT), and other transformer-based language models.

In operation, an LLM receives an input comprising text data (referred to as a "prompt") and processes the prompt through its neural network architecture to generate an output response. The output may comprise generated text, classifications, embeddings, or other representations based on the input prompt and the task for which the LLM has been configured. The LLM may be fine-tuned or adapted for specific tasks or domains through additional training on task-specific or domain-specific datasets.

Natural language processing tasks that may be performed by an LLM include, but are not limited to: text generation, language translation, text summarization, question answering, sentiment analysis, named entity recognition, text classification, conversational dialogue generation, and code generation. The LLM may operate as a standalone system or may be integrated with other components, such as retrieval systems, knowledge bases, or task-specific modules, to perform specialized functions.

The use of an LLM, and in particular, a gLM in the present invention integrates any recited abstract concepts into a practical application that transforms the claimed invention beyond a mere mental process, thereby rendering it patent-eligible subject matter under 35 U.S.C. § 101. While certain cognitive activities, such as language comprehension, information analysis, and response formulation, could theoretically be performed by the human mind using pen and paper, the scale, complexity, and computational requirements of LLM (or gLM) operations cannot practically be performed in the human mind. Specifically, a gLM/LLM processes input prompts by transforming natural language text into high-dimensional token embeddings comprising numerical vectors in multi-dimensional feature spaces, wherein each token may be represented by hundreds or thousands of numerical parameters. The gLM/LLM then performs massively parallel matrix operations across billions of model parameters using self-attention mechanisms that simultaneously evaluate contextual relationships between all tokens in the input sequence. These operations involve computing attention scores through mathematical transformations including but not limited to scaled dot-product calculations, softmax normalizations, and weighted summations across multiple attention heads and transformer layers, generating intermediate representations that are subsequently decoded to produce output tokens. The human mind is not equipped to maintain, manipulate, or process billions of numerical parameters, perform simultaneous multi-dimensional matrix calculations across vast parameter spaces, or execute the complex mathematical operations inherent in transformer architectures operating on high-dimensional vector embeddings. Furthermore, the claimed invention improves the functioning of computer systems and/or provides a technological solution to a technical problem by being capable of multi-domain, multi-protein design. Existing protein design tools are not capable of learning domain-domain, protein-protein interactions (PPIs), because they are trained on single proteins with limited context length. To date, there is no protein sequence model that is capable of generating multi-domain, multi-protein sequences at once. The present invention

is designed to be chemistry-aware. The disclosed model has a latent understanding of biochemistry by learning the relationship between chemical products and the substrate and stereo-specificities of the catalytic modules encoded in the corresponding biological sequences. In doing so, desired chemistry can serve as the conditioning signal for biosynthetic assembly line generation. The present invention provides a technical leap for the field of generative AI for chemistry, by providing an avenue to synthesize diverse sets of previously unsynthesizable molecules.

In certain embodiments, the gLM decoder is transformer-based, having 650M parameters with 20 heads, 33 layers, and 16K token context length. Other configurations will be apparent to one skilled in the art given the benefit of this disclosure.

As used herein, the term "target desired chemistry" refers to a specification of chemical characteristics that serves as input to the CABAL decoder. Target desired chemistry may include, but is not limited to, molecular structures, enzymatic domain specifications, substrate specificities, stereo-specificities, or existing biosynthetic assembly lines requiring optimization or modification. The target desired chemistry provides the conditioning signal upon which the CABAL decoder generates a corresponding biosynthetic assembly line design.

As used herein, the term "latent understanding of biochemistry" refers to learned internal representations within the neural network that encode relationships between chemical structures and protein sequences. These internal representations are acquired through training on paired datasets of chemical products and their corresponding biosynthetic machinery. The latent understanding enables the model to generate protein sequences that correspond to specified chemical characteristics without explicit programming of biochemical rules.

As used herein, a biosynthetic assembly line design comprises one or mor protein sequences. The biosynthetic assembly line is described as "capable of catalyzing" a chemical compound or enzymatic reaction when the biosynthetic assembly line, upon expression in a suitable host organism, encodes enzymatic machinery that can perform the specified chemical transformation under appropriate reaction conditions. The capability of catalysis may be

validated through experimental expression and product detection, or predicted through computational methods such as structure prediction and active site analysis.

As used herein, when a decoder or model is described as "configured to learn," this means the model architecture and training procedure are designed such that the model develops internal representations encoding the specified relationships during training. The configuration encompasses the selection of training data, model architecture, and training objectives that together enable the model to acquire the specified capabilities.

As used herein, the term "structural plausibility" refers to computational predictions indicating that a designed biosynthetic assembly line comprises one or more protein sequences that are likely to fold into a stable three-dimensional structure capable of performing its intended function. Structural plausibility may be assessed using protein structure prediction tools, including but not limited to AlphaFold, ESMFold, or similar computational methods that predict protein folding from amino acid sequences.

As used herein, "structure-based quantification of stability and foldability" refers to computational metrics derived from predicted protein structures. Such metrics may include, but are not limited to, predicted local distance difference test (pLDDT) scores, predicted aligned error (PAE), interface predicted template modeling (ipTM) scores, and free energy calculations. These metrics provide quantitative assessments of whether a designed protein sequence is likely to fold correctly and maintain structural stability.

As used herein, the identification of "fusion sites, linker sequences, and catalytic modules" by the BAL-decoder refers to the model's ability to generate sequences that include appropriate junction regions between domains (fusion sites), flexible peptide sequences connecting functional domains (linker sequences), and protein regions responsible for catalytic activity (catalytic modules). The BAL-decoder learns to generate these elements based on patterns present in the training data comprising biosynthetic assembly line sequences, enabling the generation of novel sequences that maintain proper domain organization and connectivity.

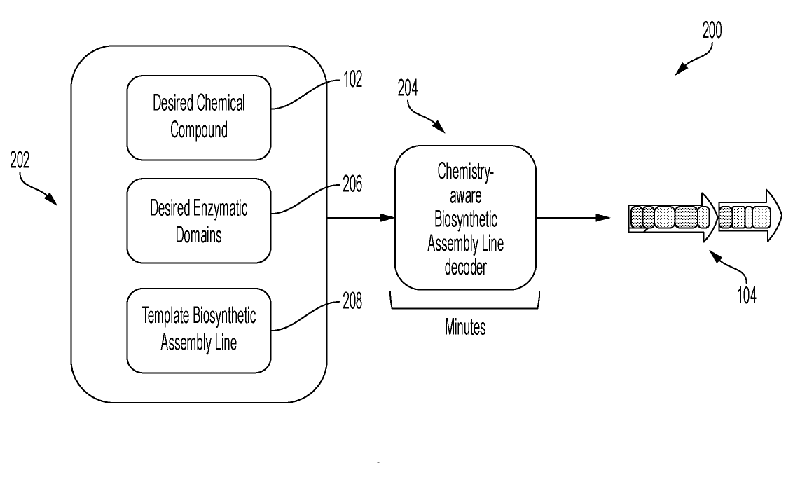

FIGS. 2 through FIG. 6 wherein like parts are designated by like reference numerals throughout, illustrate an example embodiment or embodiments of designing

biosynthetic assembly lines for producing chemical compounds using genomic artificial intelligence, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

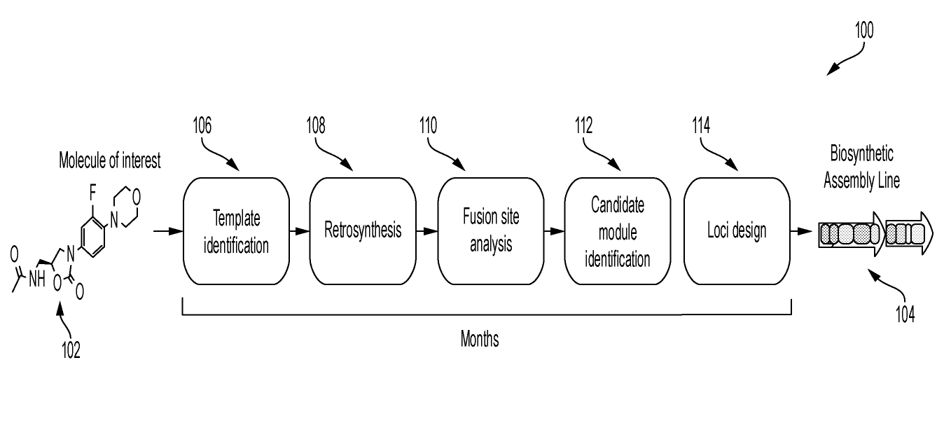

FIG. 2 depicts a high-level diagram 200 of the process of the present invention. Here, the use of a chemical to protein model 204 replaces step 106 through step 114 of the conventional process 100 shown in FIG. 1 that can generate and output a biosynthetic assembly line design 104 based on a provided target desired chemistry 202.

The target desired chemistry 202 can comprise one or more of: a desired chemical compound 102, and/or desired enzymatic domains with their substrate and stereo-specificities 206, and a template biosynthetic assembly line 208 that needs to be optimized or modified. In embodiments where the target desired chemistry 202 includes a desired chemical compound 102, the generated biosynthetic assembly line design 104 is capable of catalyzing the desired chemical compound 102. In embodiments where the target desired chemistry 202 includes desired enzymatic domains with their substrate and stereo-specificities 206, the generated biosynthetic assembly line design 104 comprises the enzymatic domains with their substrate and stereo-specificities 206. In embodiments where the target desired chemistry 202 includes a template biosynthetic assembly line 208 that needs to be optimized or modified, the generated biosynthetic assembly line design 104 is an optimized or modified biosynthetic assembly line.

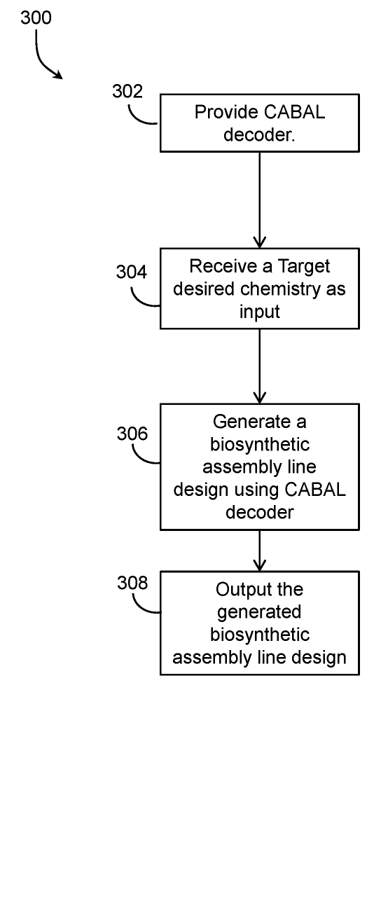

FIG. 3 depicts a flow diagram 300 for a method of designing biosynthetic assembly lines. The method comprises providing a chemistry-aware biosynthetic assembly line (CABAL) decoder 204 comprising a chemical-to-protein model configured to design a biosynthetic assembly line conditioned upon the a target desired chemistry (step 302); receiving a target desired chemistry as input (step 304); generating, using the CABAL decoder 204, a biosynthetic assembly line (step 306); and outputting the generated biosynthetic assembly line design 104 (step 308).

In certain embodiments, the chemistry-aware biosynthetic assembly line (CABAL) decoder 204 is configured for multi-domain and multi-protein design. In some embodiments, the CABAL decoder 204 is trained to comprise a latent understanding of biochemistry by learning a relationship between chemical products and catalytic modules.

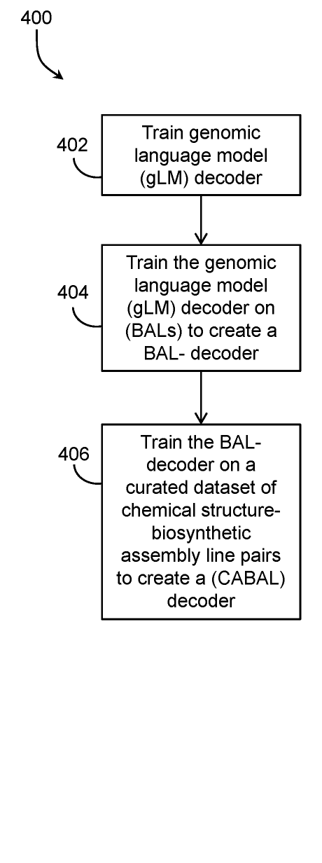

FIG. 4 depicts a flow diagram 400 for a method of generating a chemistry-aware biosynthetic assembly line (CABAL) decoder 204. The method 400 comprises training a genomic language model (gLM) decoder 510 to learn long-range logic and evolutionary constraints across multiple domains and proteins to generate multi-domain, multi-protein loci (step 402); training the genomic language model (gLM) decoder on biosynthetic assembly lines (BALs) to learn evolutionary patterns that are specific to biosynthetic assembly to create a BAL- decoder (step 404); and training the BAL-decoder on a curated dataset of chemical structure-biosynthetic assembly line pairs allowing for conditioning of sequence generation with target desired chemistry to create a chemistry-aware biosynthetic assembly line (CABAL) decoder 204(step406).

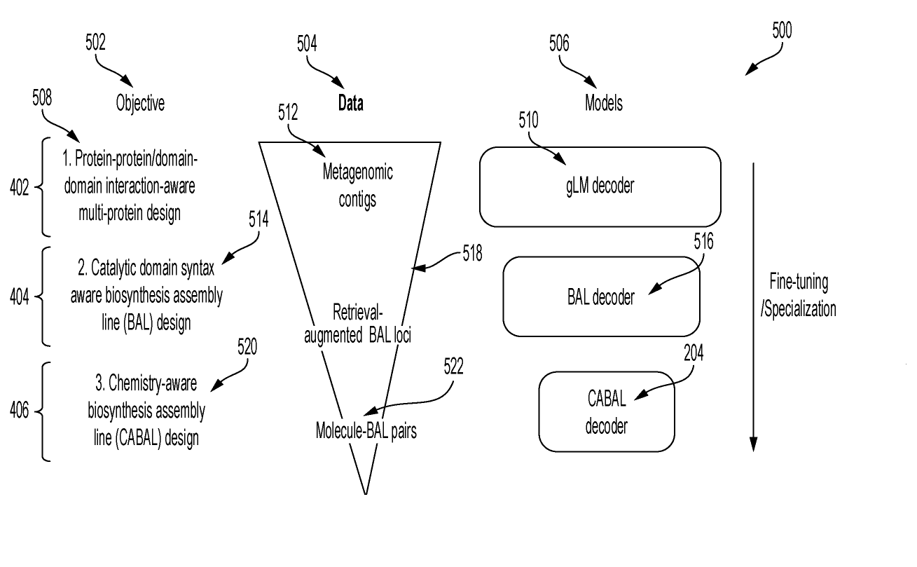

FIG. 5 is a diagram 500 depicting an example of the hierarchical training of the generative artificial intelligence (A.I.) models with an increasing degree of specialization as set forth in FIG. 4 showing the objectives 502, the training data 504, and involved models 506 in each step of the training involved in creating a CABAL decoder 204.

At the top level of the diagram 500, corresponding to step 402 of FIG. 4, the objective 508 is to train a gLM-decoder 510 about protein-protein/domain-domain interaction-aware multi-protein design. In some such embodiments, the genomic language model (gLM)-decoder 510 is trained on a multi-modal metagenomic corpus 512. As used here multi-modal means coding sequences are represented in amino acids and non-coding sequences are represented in nucleic acids. An example of such a multi-modal metagenomic corpus 512 is the Open MetaGenomic (OMG) corpus, consisting of over 3.3Tbp metagenomic sequences. Other possible data sets will be apparent to one skilled in the art given the benefit of this disclosure.

The performance of the resulting gLM-decoder 510 may be validated using in silico protein design validation methods, by structure-based quantification of stability and foldability of protein-protein complexes and domain-domain interactions.

At the next level of the diagram 500, corresponding to step 404 of FIG. 4, the objective 514 is to fine-tune (train) the gLM-decoder 510 about catalytic domain syntax aware biosynthetic assembly line (BAL) design to create a BAL-decoder 516. In certain embodiments, the gLM decoder 510 is fine-tuned on the BiG-FAM database consisting of 1,225,071 biosynthetic gene clusters. The catalytic domains are annotated for each gene cluster, and BAL decoder is trained on domain annotations - sequence pairs to allow for domain-specific conditioning in sequence decoding. The architecture of the BAL-decoder 516 is identical to the gLM decoder 510. The total parameter count is 650M. Other possible techniques and configurations will be apparent to one skilled in the art, given the benefit of this disclosure.

The objective of fine-tuning is to learn evolutionary patterns that are specific to biosynthetic assembly. In certain embodiments, the BAL-decoder 516 is configured to identify fusion sites, linker sequences, and catalytic modules and their substrate and stereo-specificities. In some embodiments, the gLM-decoder 510 is trained using retrieval-augmented BAL loci 518. Such retrieval-augmented BAL loci 518 can be provided as part of the multi-modal metagenomic corpus 512.

The resulting BAL-decoder 516 may be validated using predicted structural plausibility and detection of catalytic domains and their substrate and stereo-specificities. In certain embodiments, structural plausibility is measured using pLDDT and interface PAE scores. Catalytic domains are detected using hidden markov models of domains (e.g., using Interpro). Evaluation can be conducted using held out set of BGCs that are generated upon conditioning with desired domains. The presence and order of the detected domains is examined in the generated sequence. Substrate -specificities are evaluated by determining if known substrate specific-residues are present and further verified using in silico docking experiments. Stereo-specificity is evaluated using conserved motif analysis. Other techniques will be apparent given the benefit of this disclosure.

At the bottom level of the diagram 500, corresponding to step 406 of FIG. 4, the objective 520 is to fine-tune (train) the BAL-decoder 516 about chemistry-aware biosynthesis assembly line (CABAL) design to create the CABAL-decoder 204. The BAL-decoder 516 is fined tuned on a curated dataset of chemical structure - biosynthetic assembly line pairs 522. In certain embodiments, the chemical structure - biosynthetic assembly line pairs are provided in a SMILES representation. This final fine-tuning step allows for the conditioning of sequence generation with a target desired chemistry 202. The resulting model is a chemistry-aware BAL-decoder (CABAL-decoder 204). In certain embodiments, the CABAL-decoder 204 is configured to learn the relationship between chemical structures and biosynthetic module arrangements.

In certain embodiments, the CABAL-decoder 204 has the same architecture as BAL-decoder 516 except it is fine-tuned with SMILE representation + predicted catalytic domains to sequence mapping. The input is desired product SMILE representation with an optional list of corresponding catalytic domains and the output is the generated sequence. The SMILE representation enables the user to specify more granular details of desired product that cannot be specified by the list of desired catalytic domains.

The CABAL-decoder 204 may be validated using a curated held-out validation set.

The disclosed process 200 for designing biosynthetic assembly lines yields three generative biosynthetic assembly line models: gLM-decoder 510, Biosynthetic Assembly Line (BAL)-decoder 516 and CABAL (Chemistry-aware BAL)-decoder 204. Each of these models 510, 516, 204 are capable of protein design tasks that are impossible using current methods. In addition, the disclosed process 200 yields two major curated datasets for large scale modeling of biosynthetic machinery: 1) the retrieval augmented biosynthetic assembly line dataset 518 used for training BAL-decoder and 2) the train-test split paired-dataset of biosynthetic assembly lines and chemical products 522.

In certain embodiments, all three models are transformer encoder optimized using AdamW and trained in mixed precision bfloat16. In some such embodiments, AdamW betas are set to (0.9, 0.95) and weight decay of 0.1. Dropout is disabled throughout training.

The learning rate is warmed up for 1k steps, followed by a cosine decay to 10% of the maximum learning rate. gLM2 uses RoPE position encoding, SwiGLU feed-forward layers, and RMS normalization. Flash Attention 2 is leveraged to speed up attention computation over the sequence length of 4096.

In some embodiments, chemical structure-biosynthetic assembly line pairs are curated from the MIBiG database that contain thousands of such pairs. For training the dataset is balanced by sampling from clustered set of biosynthetic gene cluster sequences or clustered set of chemicals. Clustering is done by calculating embedding distances between BGC sequences or by chemical similarity metric (e.g., Tanimoto coefficient). When training BAL, the dataset should be around 10^6-7 examples, and for CABAL 10^3-4. Retrieval augmented for BAL generation is done first by embedding all BGCs in the database using a gLM and then retrieving similar BGCs to the query BGC in the embedding space. Train -test split is determined using the chemicals, where the train and test sets have different chemicals (with different structures). The model is evaluated by its ability to generalize to unseen chemical products.

In one experiment, BAL decoder generated Polyketide synthase designs conditioned on a set of desired domains were validated and the resulting designs yield higher product titers in the lab, when compared to rationally designed (stitched together set of domains from different organisms) assembly lines.

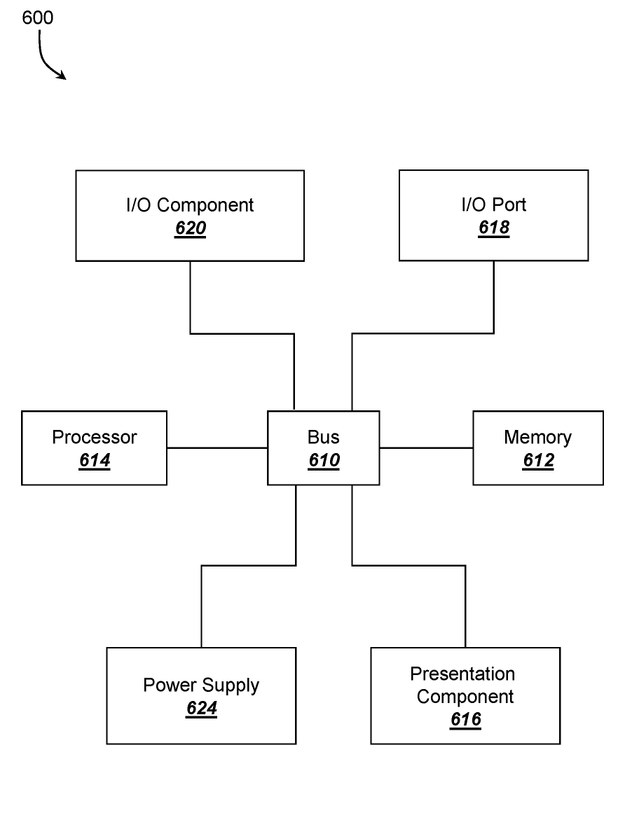

A suitable and specifically configured electronic or computing device can be used to implement systems with functionality of the present invention described herein. One illustrative example of such an electronic or computing device 600 is depicted in FIG. 6. The computing device 600 is merely an illustrative example of a suitable computing environment and in no way limits the scope of the present invention. A “computing device,” as represented by FIG. 6, can include a “workstation,” a “server,” a “laptop,” a “desktop,” a ”device,” a “smart device,” a “tablet,” a “smartphone,” an “ECR” or other specifically configured computing devices having sufficient computative processing resources to implement the invention, as would be understood by those of skill in the art. Given that the computing device 600 is depicted for illustrative purposes, embodiments of the present invention may utilize any number of computing devices 600 in any number of different ways to implement a single embodiment of the present invention. Accordingly, embodiments of the present

invention are not limited to a single computing device 600, as would be appreciated by one with skill in the art, nor are they limited to a single type of implementation or configuration of the example computing device 600.

The computing device 600 can include a bus 610 that can be coupled to one or more of the following illustrative components, directly or indirectly: a memory 612, one or more processors 614, one or more presentation components 616, input/output ports 618, input/output components 620, and a power supply 624.

One of skill in the art will appreciate that the bus 610 can include one or more buses, such as an address bus, a data bus, networks, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 6 is merely illustrative of an exemplary computing device that can be used to implement one or more embodiments of the present invention and in no way limits the invention.

The computing device 600 can include or interact with various computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD), Solid State Drive(SSD), cloud, or other optical or holographic media; magnetic cassettes, magnetic tape, magnetic disk storage or other magnetic storage devices that can be used to encode information and can be accessed by the computing device 600.

The memory 612 can include computer-storage media in the form of volatile and/or nonvolatile memory for holding data. The memory 612 may be removable, non-removable, or any combination thereof. Exemplary hardware devices are devices such as hard drives, solid-state memory, optical-disc drives, and the like. The computing device 600 can include one or more processors that read data from components such as the memory 612, the various I/O components 620, etc. Presentation component(s) 616 present data indications to a user or other device. Exemplary presentation components include a display device, speaker, printing component, vibrating component, etc.

The I/O ports 618 can enable the computing device 600 to be logically coupled to other devices, such as I/O components 620, using serial, parallel, or network and/or wireless communication protocols. Some I/O components 620 can be built into the computing device 600. Examples of such I/O components 620 include a microphone, joystick, recording device, gamepad, satellite dish, scanner, printer, wireless device, networking device, and the like.

The present invention presents the application of genomic language modeling and generative AI to design biosynthetic pathways to result in novel biochemistry. This allows for the design complicated pathways that have eluded human characterization. The presented machine learning methods learn evolutionary patterns and syntax of chemistry which can be used directly to inform design.

To any extent utilized herein, the terms “comprises” and “comprising” are intended to be construed as being inclusive, not exclusive. As utilized herein, the terms “exemplary,” “example,” and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may exist in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near

complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method of designing biosynthetic assembly lines, the method comprising: providing a chemistry-aware biosynthetic assembly line (CABAL) decoder comprising a chemical-to-protein model configured to design a biosynthetic assembly line conditioned upon a target desired chemistry; receiving a target desired chemistry as input; generating, using the CABAL decoder, a biosynthetic assembly line design ; and outputting the generated biosynthetic assembly line design.

2. The method of claim 1, wherein the chemistry-aware biosynthetic assembly line (CABAL) decoder is configured for multi-domain and multi-protein design.

3. The method of claim 1, wherein the CABAL decoder is trained to comprise a latent understanding of biochemistry by learning a relationship between chemical products and catalytic modules.

4. The method of claim 1, wherein the provided chemistry-aware biosynthetic assembly line (CABAL) decoder is created by: training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder.

5. The method of claim 4, wherein the genomic language model (gLM) decoder is trained on a multi-modal Open MetaGenomic (OMG) corpus.

6. The method of claim 4, wherein performance of gLM-decoder is validated using in silico protein design validation methods, by structure-based quantification of stability and foldability of protein-protein complexes and domain-domain interactions.

7. The method of claim 4, wherein the BAL-decoder is configured to identify fusion sites, linker sequences, and catalytic modules and their substrate and stereo-specificities.

8. The method of claim 4, wherein the BAL-decoder is validated using predicted structural plausibility and detection of catalytic domains and their substrate and stereo-specificities.

9. The method of claim 4, wherein the chemistry-aware biosynthetic assembly line (CABAL) decoder is configured to learn relationships between chemical structures and biosynthetic module arrangements.

10. The method of claim 4, wherein the chemistry-aware biosynthetic assembly line (CABAL) decoder is validated using a curated held-out validation set.

11. The method of claim 1, wherein the target desired chemistry comprises a desired chemical compound and the generated biosynthetic assembly line design is capable of catalyzing the desired chemical compound.

12. The method of claim 1, wherein the target desired chemistry comprises desired enzymatic domains with their substrate and stereo-specificities and the generated biosynthetic assembly line design comprises the enzymatic domains with their substrate and stereo-specificities.

13. The method of claim 1, wherein the desired target chemistry comprises a template biosynthetic assembly line that needs to be optimized or modified and the generated biosynthetic assembly line design comprises an optimized or modified biosynthetic assembly line.

14. The method of claim 1, further comprising training the chemical-to-protein model using one or more of: a retrieval augmented biosynthetic assembly line dataset and a train-test split paired-dataset of biosynthetic assembly lines and chemical products.

15. A system for designing biosynthetic assembly lines, the system comprising: a chemistry-aware biosynthetic assembly line (CABAL) decoder comprising a chemical-to-protein model configured to design a biosynthetic assembly line conditioned upon a target desired chemistry ; and a processor configured to: receive a target desired chemistry as input; generate, using the CABAL decoder, a biosynthetic assembly line ; and output the generated biosynthetic assembly line design.

16. The system of claim 15, wherein the chemistry-aware biosynthetic assembly line (CABAL) decoder is configured for multi-domain and multi-protein design.

17. The system of claim 15, wherein the CABAL decoder is trained to comprise a latent understanding of biochemistry by learning a relationship between chemical products and catalytic modules.

18. The system of claim 15, wherein the chemistry-aware biosynthetic assembly line (CABAL) decoder is created by: training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder.

19. The system of claim 18, wherein the genomic language model (gLM) decoder is trained on a multi-modal Open MetaGenomic (OMG) corpus.

20. The system of claim 18, wherein performance of gLM-decoder is validated using in silico protein design validation methods, by structure-based quantification of stability and foldability of protein-protein complexes and domain-domain interactions.

21. The system of claim 18, wherein the BAL-decoder is configured to identify fusion sites, linker sequences, and catalytic modules and their substrate and stereo-specificities.

22. The system of claim 18, wherein the BAL-decoder is validated using predicted structural plausibility and detection of catalytic domains and their substrate and stereo-specificities.

23. The system of claim 18, wherein the chemistry-aware biosynthetic assembly line (CABAL) decoder is configured to learn relationships between chemical structures and biosynthetic module arrangements.

24. The system of claim 18, wherein the chemistry-aware biosynthetic assembly line (CABAL) decoder is validated using a curated held-out validation set.

25. The system of claim 15, wherein the target desired chemistry comprises a desired chemical compound and the generated biosynthetic assembly line design is capable of catalyzing the desired chemical compound.

26. The system of claim 15, wherein the target desired chemistry comprises desired enzymatic domains with their substrate and stereo-specificities and the generated biosynthetic assembly line design is capable of catalyzing the enzymatic domains with their substrate and stereo-specificities.

27. The system of claim 15, wherein the desired target chemistry comprises a template biosynthetic assembly line that needs to be optimized or modified and the generated biosynthetic assembly line design comprises an optimized or modified biosynthetic assembly line.

28. The system of claim 15, wherein the processor is further configured to train the chemical-to-protein model using one or more of: a retrieval augmented biosynthetic assembly line dataset and a train-test split paired-dataset of biosynthetic assembly lines and chemical products.

29. A method of generating a chemistry-aware biosynthetic assembly line (CABAL) decoder, the method comprising: training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder.

30. The method of claim 29, wherein the genomic language model (gLM) decoder is trained on a multi-modal Open MetaGenomic (OMG) corpus.

31. The method of claim 29, wherein performance of gLM-decoder is validated using in silico protein design validation methods, by structure-based quantification of stability and foldability of protein-protein complexes and domain-domain interactions.

32. The method of claim 29, wherein the BAL-decoder is configured to identify fusion sites, linker sequences, and catalytic modules.

33. The method of claim 29, wherein the BAL-decoder is validated using predicted structural plausibility and detection of catalytic domains and their substrate and stereo-specificities.

34. The method of claim 29, wherein the chemistry-aware biosynthetic assembly line (CABAL) decoder is configured to learn relationships between chemical structures and biosynthetic module arrangements.

35. The method of claim 29, wherein the chemistry-aware biosynthetic assembly line (CABAL) decoder is validated using a curated held-out validation set.

36. The method of claim 29, wherein target desired chemistry comprises one or more of a desired chemical compound, desired enzymatic domains with their substrate and stereo-specificities, and a template biosynthetic assembly line that needs to be optimized or modified.

37. A chemical-to-protein model configured to design a biosynthetic assembly line conditioned upon a target desired chemistry, the chemical-to-protein model created by a process comprising: training a genomic language model (gLM) decoder to learn long-range logic and evolutionary constraints across multiple domains and proteins to generate multi-domain, multi-protein loci; training the genomic language model (gLM) decoder on biosynthetic assembly lines (BALs) to learn evolutionary patterns that are specific to biosynthetic assembly to create a BAL-decoder; and training the BAL-decoder on a curated dataset of chemical structure-biosynthetic assembly line pairs allowing for conditioning of sequence generation with target desired chemistry to create a chemistry-aware biosynthetic assembly line (CABAL) decoder.

38. The chemical-to-protein model of claim 37, wherein the genomic language model (gLM) decoder is trained on a multi-modal Open MetaGenomic (OMG) corpus.

39. The chemical-to-protein model of claim 37, wherein performance of gLM-decoder is validated using in silico protein design validation methods, by structure-based quantification of stability and foldability of protein-protein complexes and domain-domain interactions.

40. The chemical-to-protein model of claim 37, wherein the BAL-decoder is configured to identify fusion sites, linker sequences, and catalytic modules and their substrate and stereo-specificities.

41. The chemical-to-protein model of claim 37, wherein the BAL-decoder is validated using predicted structural plausibility and detection of catalytic domains and their substrate and stereo-specificities.

42. The chemical-to-protein model of claim 37, wherein the chemistry-aware biosynthetic assembly line (CABAL) decoder is configured to learn relationships between chemical structures and biosynthetic module arrangements.

43. The chemical-to-protein model of claim 37, wherein the chemistry-aware biosynthetic assembly line (CABAL) decoder is validated using a curated held-out validation set.

44. The chemical-to-protein model of claim 37, wherein target desired chemistry comprises one or more of a desired chemical compound, desired enzymatic domains with their substrate and stereo-specificities, and a template biosynthetic assembly line that needs to be optimized or modified.
